(19) United States
(12) Patent Application Publication
(10) Pub. No.: US 2006/0014284 A1
(43) Pub. Date: Jan. 19, 2006

(54) Biomatrix and method for producting the same

(76) Inventor: Thomas Graeve, Suttgart (DE)

Correspondence Address: WILLIAM COLLARD;COLLARD & ROE, P.C., 1077 NORTHERN BOULEVARD, ROSLYN, NY, 11576, US

(21) Appl. No.: 10/508,224

(22) PCT Filed: Mar. 19, 2003
(86) PCT No.: PCT/DE03/00904

§ 371 Date: Aug. 24, 2005

Related U.S. Application Data
(60) Provisional application No. 60/409,961, filed on Sep. 11, 2002.

(30) Foreign Application Priority Data
Mar. 21, 2002 (DE) 102 12 715.8
Jul. 22, 2002 (DE) 102 33 291.6
Sep. 6, 2002 (DE) 102 41 817.9

(51) Int. Cl. C12N 5/00

(52) U.S. Cl. 435/397; 514/2

(57) ABSTRACT

The invention relates to a biomatrix as well as to a method of manufacturing same. In order to provide a biologically and mechanically satisfying replacement for diseased cartilage for example, a biomatrix is proposed in which the matrix is condensed or solidified. A corresponding method is moreover proposed.

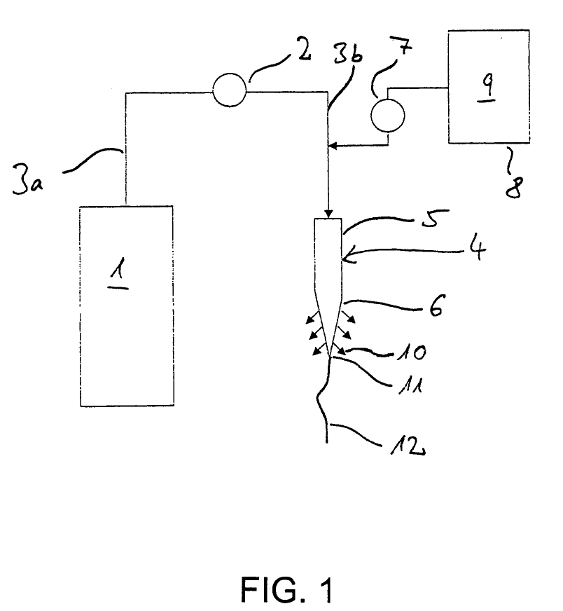

The invention relates to a biomatrix and to a method of manufacturing same.

In medical engineering, more specifically in surgery, one regularly faces the task of removing defective constituent parts of the human or animal body and of replacing them with ones that are artificially manufactured. Intensive research is hereby conducted with regard to medical engineering. It has been found that many special solutions are needed for the many different task fields.

In the field of repairing joint disease, the document DE 100 26 789 A1 for example describes cartilage replacement permitting good regeneration of a treated cartilage defect. The application mentioned hereby makes use of a 3D biomatrix that may conform to the shape and size of the defect to be replaced.

Many other proposals have been made in the art regarding implants in the field described. The materials used in this case are either three-dimensional, but porous, for example when they are configured to be a sponge or a fleece; or they are solid but have no three-dimensional structure, for example when they are configured to be a foil.

Specific demands have to be placed on implants that are to replace parts of the mechanical structure of the body, though. On the one side, a defect to be repaired generally has at least a certain three-dimensionality so that the implant should have a three-dimensional shape as well. On the other side however, the implant must be capable of taking into account, by its density and more specifically by its pressure stability, the mechanical loads to which it is subjected when used in the structure of the body. More specifically, the pressure stability of the prior art materials is not sufficient for use in cartilage for example.

The object of the inventor therefore is to improve a biomatrix in such a manner that the biomatrix as an implant is capable of mechanically taking the high loads generated. It is another object of the inventor to develop a method by means of which such a biomatrix may be manufactured.

The solution to this object is surprisingly well achieved by a biomatrix in which the matrix is condensed or solidified.

A collagen biomatrix as it is described in the document DE 100 26 789 A1 may be three-dimensional in itself. By condensing or solidifying the collagen biomatrix, the biological material becomes so solid that the dynamometric requirements in the mechanical structure of the body may be met. The pressure stability of the condensed or solidified material in particular can meet very high demands.

But the tensile strength is also strongly increased over prior art biomatrices as a result of increasing resistance to transverse contraction which comes along with the increased pressure stability.

Accordingly, the invention allows for extensive possibilities of utilization for implants, for example as cartilage replacement with high pressure stability, ligament replacement, tendon replacement, meniscus replacement, intervertebral disc replacement, nucleus replacement and/or annulus replacement. Such an implant may even be utilized for bone replacement or as a replacement for bone/cartilage structures.

The biomatrix condensed or solidified in accordance with the invention may advantageously be characterized in that it comprises a density gradient. The biomatrix can be homogeneously condensed although the collagen scaffold may also be made in the form of a gradient. This makes it possible to conform in a particular manner the resulting implant to the defect of the body in accordance with the dynamometric requirements. It is further possible to economically manufacture implants with reinforced regions for particular requirements.

In a preferred variant thereof, the density gradient is configured to be stepped. A biomatrix in accordance with the invention is quite easy to manufacture regularly with a stepped density gradient.

Alternatively, the density gradient may also comprise a continuous transition. Although a biomatrix that is implemented in this manner is quite complicated to manufacture, it may be perfectly adapted to the mechanical requirements at a defect site. Thanks to a continuous density gradient profile, an implant can be inserted particularly homogeneously into the surrounding body structure without damaging tension peaks being generated as this is the case with discontinuous density gradients of the implant and continuously changing properties of the neighboring structures.

The biomatrix of the invention may distinguish itself more preferably in that the density gradient forms a body with a more solid outer shell and a softer core. As used herein, a softer core also means a much softer core, and also a core without an inherent hardness. The core may for example be filled with a fluid or more specifically with a gas so that a mechanical effect similar to that of an air cushion is achieved. As a result, the properties of the implant may be particularly well matched to the neighboring structures. The soft core provides for sufficient flexibility of the biomatrix in the inserted condition, whereas the solid outer shell provides a particularly good protection from mechanical degradation of the biomatrix. Accordingly, the biomatrix, while having a long life and featuring high robustness, can still exhibit sufficient elastic behavior.

It is to be understood that a biomatrix with a density gradient is advantageous and inventive irrespective of explicit condensation or solidification of the biomatrix.

Independently thereof, it is advantageous if the matrix of the invention is combined with inert materials. The inert materials may hereby be of a biological or nonbiological origin. Collagen fleeces will be mentioned here by way of example.

In a preferred embodiment, the biomatrix of the invention comprises oriented fibers, more specifically collagen fibers. The collagen fibers may be oriented in the matrix by combining condensation and simultaneous application of alternating pressure and tensile load. Alternatively and cumulatively, this is also possible by applying alternating physical stresses or by using other physical methods. The oriented fibers advantageously affect the physical properties of the biomatrix in particular. If the fibers used therefore are collagen fibers, both the physical and the biochemical properties are considerably improved.

It is to be understood that the presence of oriented fibers, more specifically of collagen fibers, in the biomatrix may also be achieved without explicit condensation or solidification and that such a presence is advantageous and inventive irrespective thereof.

Eventually, an implementation variant of the biomatrix, of the invention is particularly preferred in which the initial solution comprises 1 to 10 mg of protein for each ml of gel solution and this initial solution is condensed to more than ten times, preferably more than fifty times the initial protein content. During condensation, the initial protein content may even be increased a hundredfold. A high pressure-stable matrix of high solidity is thus achieved.

The objective of the invention is also accomplished by a method of manufacturing a biomatrix by which the matrix is condensed or solidified. By condensing or solidifying the biomatrix, it achieves the above mentioned advantageous properties.

The method of the invention is preferably characterized in that the matrix is manufactured by a method for redifferentiating and/or growing dedifferentiated cartilage cells by which dedifferentiated cartilage cells that are embedded in a three-dimensional, gel-like biomatrix containing at least 1.5 mg/l of collagen in the buffered serum-containing cell growth medium are cultivated. The major problem of degenerative or post-traumatic joint diseases is that the damaged joint cartilage exhibits but little capacity for regeneration. Inasmuch, it is a major object to durably introduce with the implant cartilage cells that allow for regeneration of the cartilage to the site of the defect. When the matrix is manufactured by the method described, the dedifferentiated cartilage cells in the biomatrix are capable of redifferentiating and of resuming their cell-specific metabolic performances. For this purpose, the biomatrix should contain a collagen scaffold newly constituted from a preferably fresh collagen solution and having a concentration of at least 1.5 mg of collagen for each ml of biomatrix.

As used herein, the term cultivation of cells means maintaining, preferably in vitro, the life functions of cells in a suited environment, for example by supplying and evacuating metabolic educts and products, more specifically also the growth of the cells. The term cartilage cells is meant to include naturally occurring or genetically modified cartilage cells or their precursors that may be of animal or human origin.

In a particularly preferred variant, the method of the invention is characterized in that the matrix is manufactured by a method including the isolation of collagen-containing tissue, the transfer of the collagen-containing tissue into an acidic solution, the incubation of the collagen tissue transferred into the acidic solution at 2 to 10° C., more specifically at 4° C., the removal by centrifugation of undissolved collagen fractions, the mixing of the collagen solution obtained at 2 to 10° C., preferably at 4° C., with a solution containing cell growth medium, serum and buffer and the gelification of the mixed solution by increasing the temperature. This method is very effective and is capable of providing sufficient cell material for manufacturing cartilage transplants even if the initial quantity of cartilage tissue is low. Additionally, homogeneous distribution of the cells in the biomatrix is achieved in a simple and reliable manner. The solidity of the resulting material is hereby adjustable by the initial volume of the collagen solution.

Depending on the application, it may be advantageous if, in the method of the invention, the matrix is manufactured with cells. The condensed or solidified material can be manufactured with or without cells. Manufacturing the implant with cells promotes its biocompatibility in the manner described.

In an alternative, the matrix is manufactured without cells with the method of the invention. This is particularly opportune when no suited cells are available for manufacturing the matrix. Additionally, the resulting implant is simpler and less expensive to manufacture while equally meeting the requirements placed on mechanical strength.

With the method of the invention it is advantageous to effect condensation by liquid removal. If the matrix has liquid in excess, removal of this excess liquid is the simplest possibility to condense or solidify the matrix. There are many possibilities to put this into practical effect. Physically, the matrix may for example be pressurized or a negative pressure may be applied. It is also possible to reliably remove liquid through capillary forces or through a combination of the possibilities described. In most cases, this will be the simplest way of removing water. Such a liquid removal can be particularly efficiently achieved by pressurization or by application of a negative pressure.

Alternatively and cumulatively, it is advantageous if the condensation is effected by a chemical bond. A chemical bond is another possibility to remove water and/or another liquid from the biomatrix and to thus condense or solidify the biomatrix in order to achieve the properties desired.

Moreover, the method of the invention permits to also manufacture a collagen thread of between 0.005 and 4 mm, preferably of between 0.01 and 2 mm, in diameter. Such a collagen thread offers many new possibilities for utilization in the human or animal body. It may more specifically even have a connecting function.

For manufacturing such a thread it is proposed that the collagen solution be passed through a nozzle. The reduced cross-section in the nozzle forcibly causes the biomatrix to condense. For this purpose, the liquid collagen can be pressed with or without buffer solution through the nozzle that removes the liquid from the collagen. This may happen by applying a pressure below or above ambient pressure. After condensation, the collagen thread may be dried, with about 10% of residual moisture being perfectly admissible.

Methods in accordance with the invention are explained herein after with respect to two examples. It is desired to note, however, that the present patent application is not only related to cartilage cells like in the example, but also to bone cells, stem cells, fibroblasts, endothelial cells, muscle cells, epithelium cells, epithelial cells, glandular cells, sensory cells and to combinations of the cells mentioned. It is perfectly possible to use the invention for these kinds of cells as well.

### EXAMPLE 1

The initial material used is a collagen solution as it is described in the document DE 100 26 789 A1 and having a protein concentration of 6 mg/ml of gel solution. A buffer solution is simultaneously used for buffering the acidic collagen solution.

The two liquids are mixed together in equal portions at 4° C. The collagen concentration in the mixed solution accordingly amounts to about 3 mg/ml. This solution is now filled into a glass cylinder of about 2 cm in diameter. In the present case, about 35 ml of the collagen-buffer solution mixture are filled in the cylinder, the filling height obtained is about 10 cm.

The glass cylinder is thereby placed with its lower opening in a glass base ensuring a reliable seal. The lower end of the glass cylinder is additionally closed with a porous, liquid-permeable membrane.

The collagen-buffer solution mixture contained in the cylinder is incubated for 20 minutes at 37° C. and fully gelifies thereby. Next, a pressure piston is applied onto the gelified matrix, said pressure piston being introduced into the glass cylinder through the upper open end thereof and pressurizing the matrix along the cylinder axis. In the present case, the pressure amounts to about 0.2 Pa and is maintained for 20 minutes.

The pressure causes the matrix to be compressed and the liquid to be removed therefrom. This first occurs at the upper end of the matrix to which the pressure is initially applied; shortly thereafter, liquid also exits the lower opening closed by the liquid-permeable membrane. The pressure piston is carried along with the compressed matrix in order to maintain the applied pressure so that it remains as far as possible unreduced.

As the matrix condenses, meaning as the height of the matrix column decreases, the protein content of collagen increases and the biomatrix solidifies in accordance with the invention. After pressurization, the pressure piston is removed and the glass cylinder is taken off the base. The exiting liquid simply drains and there remains the condensed matrix. In the present test, the matrix still has a height of about 2 cm after condensation so that the protein content is five times the protein content in the initial mixture and amounts to about 15 mg/ml. If the matrix is further compressed, protein contents of collagen of for example 30 mg/ml of gel and more may perfectly well be achieved.

The condensed matrix is finally mechanically cleaned and shaped in accordance with the future application, for example cut. Then, the transplant is ready for cultivation.

The wall of the plain cylinder may thereby be made of a non-porous material, for example of glass, or of a porous material, for example of an ultrafiltration membrane. The shape of a cylinder is thereby not binding, compression or solidification of the biomatrix can be performed in any other forming configuration, for example in the shape of a ball or of a strand. The shape given to the condensed matrix is thereby function of the future application.

The condensed matrix may also be caused to conform to the future shape by mechanical methods such as cutting or stamping as well as by physical methods such as a laser. Diameter and thickness of the condensed matrix are hereby variable. The diameter may range from 1 mm to 200 mm, the thickness from 1 mm to 50 mm.

### EXAMPLE 2

To illustrate this example of a method in accordance with the invention, reference is made to the drawing. In the drawing:

the unique FIG. shows schematically an experimental set-up for manufacturing collagen fibers.

Again, a collagen solution 1 as described herein above is used as the initial material. This solution is pressed by a pump 2 via a line 3a, 3b through a nozzle 4 with a cylindrical portion 5 and a conical portion 6. Optionally, a buffer solution 9 may also be introduced by an additional pump 7 from a reservoir 8 into the line 3b and be conducted through the nozzle 4 together with the collagen solution 1.

As a result of the reduced cross-section in the conical portion 6 of the nozzle 4, the collagen solution is highly condensed with liquid being thereby removed (denoted by way of example in the FIG. by the arrows labeled at 10). A collagen thread 12 exits an exit port 11 of the nozzle 4, said collagen thread having substantially the same diameter as the exit port 11. The collagen thread 12 may then be dried.

1-20. (canceled)

21. A condensed collagen biomatrix, comprising a density gradient inside the matrix between a first range with a protein concentration between 1 and 10 mg/ml of gel solution and a second range with a protein concentration of more than 10 mg/ml, preferably of more than 50 mg/ml up to 1000 mg/ml.

22. The biomatrix according to claim 21, wherein the density gradient is configured to be stepped.

23. The biomatrix according to claim 21, wherein the density gradient comprises a continuous transition.

24. The biomatrix according to claim 21, wherein the density gradient forms a body with a more solid outer shell and a softer core.

25. The biomatrix according to claim 21, wherein the matrix is combined with inert materials.

26. The biomatrix according to claim 21, wherein it comprises oriented fibers, more specifically collagen fibers.

27. Use of a fresh collagen solution for a method of manufacturing a densified or solidified collagen biomatrix.

28. Use according to claim 27, wherein in the manufacturing method the matrix is manufactured by a method for redifferentiating and/or growing dedifferentiated cartilage cells by which dedifferentiated cartilage cells that are embedded in a three-dimensional, gel-like biomatrix containing at least 1.5 mg of collagen for each liter in buffered serum-containing cell growth medium are cultivated.

29. Use according to claim 27, wherein in the manufacturing method the matrix is manufactured by a method including the isolation of collagen-containing tissue, the transfer of the collagen containing tissue into an acidic solution, the incubation of the collagen tissue transferred into the acidic solution at 2 to 10° C., more specifically at 4° C., the removal by centrifugation of undissolved collagen fractions, the mixing of the collagen solution obtained at 2 to 10° C., preferably at 4° C., with a solution containing cell growth medium, serum and buffer and the gelification of the mixed solution by increasing the temperature.

30. Use according to claim 27, wherein in the manufacturing method the matrix is manufactured with cells.

31. Use according to claim 27, wherein in the manufacturing method the matrix is manufactured without cells.

32. Use according to claim 27, wherein in the manufacturing method condensation is effected by liquid removal.

33. Use according to claim 32, wherein liquid removal is initiated by pressurization.

34. Use according to claim 33, wherein liquid removal is executed by applying a negative pressure.

35. Use according to claim 27, wherein in the manufacturing method condensation is effected by a chemical bond.

36. A method for manufacturing a condensed collagen biomatrix, wherein a fresh collagen solution is passed through a nozzle for forming a collagen thread of between 0.005 and 4 mm, preferably of between 0.01 and 2 mm, in diameter.

37. The method according to claim 36, wherein the manufacturing method begins with an initial solution that comprises 1 to 10 mg of proteins for each ml of gel solution and that condensation is carried out up to a protein concentration of more than 10 mg/ml, preferably of more than 50 mg/ml up to 1000 mg/ml of the biomatrix.
